(19) United States
(12) Patent Application Publication
(10) Pub. No.: US 2014/0314672 A1
(43) Pub. Date: Oct. 23, 2014

(54) NANOPARTICLE THERAPEUTIC AGENTS, THEIR FORMULATIONS, AND METHODS OF THEIR USE

(71) Applicant: IGDRASOL, Inc., Fountain Vallley, CA (US)

(72) Inventor: Vuong Trieu, Agoura Hills, CA (US)

(73) Assignee: IGDRASOL, INC., Fountain Valley, CA (US)

(21) Appl. No.: 14/244,373

(22) Filed: Apr. 3, 2014

Related U.S. Application Data
(60) Provisional application No. 61/809,312, filed on Apr. 6, 2013.

(51) Int. Cl. A61K 9/51; A61K 49/00; A61K 9/107; A61K 47/48; A61K 31/337

(52) U.S. Cl. CPC A61K 9/5123; A61K 47/48723; A61K 31/337; A61K 9/1075; A61K 49/00
USPC 424/9.1; 424/490; 424/178.1; 514/449; 424/497

(57) ABSTRACT

Nanoparticle therapeutic agents, formulations that include the nanoparticle therapeutic agents, and methods for treating diseases treatable by the therapeutic agents.

### CROSS-REFERENCE TO RELATED APPLICATION

This application claims the benefit of U.S. Provisional Application No. 61/809,312, filed Apr. 6, 2013, which is expressly incorporated herein by reference in its entirety.

### FIELD OF THE INVENTION

The present invention provides nanoparticle therapeutic agents, formulations that include the nanoparticle therapeutic agents, and methods for using the nanoparticle therapeutic agents and formulations.

### BACKGROUND OF THE INVENTION

Paclitaxel is one of the most potent anticancer agents for the treatment of several cancers, including breast, ovarian, and lung cancers. Paclitaxel is a lipophilic molecule and is virtually insoluble in water. The poor aqueous solubility of paclitaxel has hindered the development of a suitable formulation for administration to patients.

The first commercially available paclitaxel product, Taxol® (Bristol-Myers Squibb Oncology), is formulated in a vehicle containing an approximately 1:1 (v/v) mixture of polyoxyethylated castor oil (Cremophor EL) and ethanol. There are several disadvantages associated with the use of Taxol®. Foremost among these is the presence of Cremophor EL in the formulation. Cremophor EL has been associated with bronchospasm, hypotension, and other manifestations of hypersensitivity, particular following rapid administration. As a result, the administration of Taxol® requires long infusion times of diluted material and premedication to reduce these adverse effects. Typically, Taxol® is diluted about 10 to 20 fold prior to administration, and the approved infusion times range from 3 to 24 hours.

Efforts have been made to provide paclitaxel formulations that overcome the problems associated with Taxol®. In one approach, the aqueous solubility of paclitaxel has been enhanced through the development of pro-drugs, such as pegylated paclitaxel or polyglutamate paclitaxel. These compounds successfully increase the aqueous solubility of paclitaxel and thereby avoid the use of toxic solvents to solubilize paclitaxel. However, the pro-drugs require the presence of enzymes in the blood or tissue to cleave the water-soluble component of the pro-drug from the paclitaxel moiety. Therefore, the therapeutic utility of paclitaxel can be compromised if the level of activity of the enzyme required to release the paclitaxel from the pro-drug is low, as is frequently the case among the cancer patients. Generally, these pro-drugs are infused slowly to avoid adverse reactions.

Another approach has used human albumin-coated paclitaxel nanoparticles (Abraxane® or nab-paclitaxel) to avoid the use of toxic solvents. Though a breakthrough in paclitaxel formulation, nab-nanoparticle has inherent problems associated with any biologic. A further related approach utilizes a chemical polymer-bound nanoparticle paclitaxel (IG-001), rather than the biological polymer-bound nab-paclitaxel. IG-001 is currently being developed as a next generation nanoparticle paclitaxel targeting difficult-to-perfuse hypoxic tumors, such as pancreatic cancer, by taking advantage of its ability to rapidly deliver paclitaxel to the targeted tissue via albumin mediated transport.

Despite the advances in the development of paclitaxel formulations noted above, there remains a need for paclitaxel formulations that overcome the disadvantages of prior art formulations. Moreover, there remains a need to identify defining characteristics of effective paclitaxel formulations to provide guidance for the design of new and improved paclitaxel formulations. The present invention seeks to fulfill this need and provides further related advantages.

### SUMMARY OF THE INVENTION

The present invention provides nanoparticle therapeutic agents, formulations that include the nanoparticle therapeutic agents, and methods for using the nanoparticle therapeutic agents and formulations.

In one aspect, the invention provides a method for identifying characteristics defining clinically successful nanoparticle therapeutic agent (e.g., paclitaxel) formulations.

In another aspect, the invention provides nanoparticle therapeutic agents, their formulations, and methods for using the formulations to treat diseases, disorders, and conditions.

In one embodiment, the invention provides a nanoparticle therapeutic agent comprising a therapeutic agent in a carrier (or vehicle). The nanoparticle of the invention has a size sufficient to be taken into and/or across a cell of interest by active transport through a cell surface organelle. Active transport includes transcytosis, particularly caveolae-mediated transcytosis, clathrin-mediated transcytosis, and clathrin- and caveolae-independent transcytosis.

In one embodiment, the nanoparticle comprises:

(a) a core comprising a therapeutic agent and a tocopherol; and

(b) a shell comprising a tocopherol polyethylene glycol derivative,

wherein the shell surrounds the core, and

wherein the nanoparticle has a size sufficient to be taken into and/or across a human cell by active transport through a cell surface organelle.

In certain embodiments, the nanoparticle further includes a targeting agent to selectively target the nanoparticle to a cell or cells of interest.

In other embodiments, the nanoparticle further includes an imaging agent.

In further embodiments, the nanoparticle includes both a targeting agent and an imaging agent.

In another aspect of the invention, an emulsion is provided. In one embodiment, the emulsion comprises a water phase; and an oil phase comprising a plurality of nanoparticles of the invention.

In a further aspect, the invention provides method for treating a disease, condition, or disorder treatable by administering a specific therapeutic agent. In one embodiment, the method includes administering a therapeutically effective amount of a nanoparticle of the invention, or an emulsion, or polymeric micelle, or formulation of the invention comprising the specific therapeutic agent, to a subject in need thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a plot of Net (Δ) Overall Response Rate (ΔORR) of Phase 3 data for IG-001 (interim), Abraxane, and Tocosol. For both IG-001 and Abraxane, improvement in ORR was observed over that of Taxol. However, no improvement was observed for Tocosol over Taxol.

FIG. 2 compares AUCinf (ng*h/mL) as a function of paclitaxel dose (mg/m²) for four paclitaxel formulations: (a) Abraxane, (b) IG-001, (c) Taxol, and (d) a paclitaxel TPGS nanoemulsion (IG-002).

FIG. 3 compares paclitaxel release from three paclitaxel formulations: (a) Abraxane, (b) Taxol, and (c) a paclitaxel TPGS nanoemulsion (IG-002) (compared to paclitaxel control).

FIG. 4 is a plot of particle size versus paclitaxel concentration for nab-paclitaxel in phosphate buffered saline (PBS) and 0.1× serum and 1× serum.

FIG. 5 is a plot of particle size versus paclitaxel concentration for IG-001 in phosphate buffered saline (PBS) and 0.1× serum and 1× serum.

FIG. 6 compares antitumor (murine B16 melanoma) activities of two paclitaxel formulations: (a) Taxol at 20 mg paclitaxel/kg and (b) a paclitaxel TPGS nanoemulsion (IG-002) at 20, 40, and 60 mg paclitaxel/kg (compared to saline and vehicle controls).

FIG. 7 compares antitumor (murine B16 melanoma) activities of two paclitaxel formulations: (a) Taxol at 20 mg paclitaxel/kg and (b) IG-001 at 20 and 50 mg paclitaxel/kg (compared to control and vehicle controls).

FIG. 8 compares antitumor (DLD-1) activities of three intravenous taxane formulations (Q4dx3): (a) Taxol at 20 mg paclitaxel/kg, (b) IG-001 (Genexol-PM) at 50 mg paclitaxel/kg, and (c) Taxotere at 13 mg/kg (compared to control).

FIG. 9 compares antitumor (NCI-H1299) activities of three intravenous taxane formulations (Q4dx3): (a) Taxol at 20 mg paclitaxel/kg, (b) IG-001 (Genexol-PM) at 50 mg paclitaxel/kg, and (c) Taxotere at 13 mg/kg (compared to control).

FIG. 10A illustrates IG-001 nanoparticles at concentrations above its critical micelle concentration (CMC) (see peak) and the absence of nanoparticles at physiological concentrations (see second arrow).

FIG. 10B illustrates Abraxane nanoparticles at concentrations above its critical micelle concentration (CMC) (see peak) and the absence of nanoparticles at physiological concentrations (see second arrow).

FIG. 11 compares paclitaxel tumor distribution for IG-002 (TocPac), Taxol, and Abraxane in a MDA-MB-435 xenograft model (dose level of 10 mg/kg).

FIG. 12 is a schematic illustration of transcytosis processes.

FIG. 13 is a schematic illustration of representative nanoparticles.

### DETAILED DESCRIPTION OF THE INVENTION

The present invention provides nanoparticle therapeutic agents, formulations that include the nanoparticle therapeutic agents, and methods for using the nanoparticle therapeutic agents and formulations.

In one aspect, the invention provides a method for identifying characteristics defining clinically successful nanoparticle therapeutic agent (e.g., paclitaxel) formulations.

In another aspect, the invention provides nanoparticle therapeutic agents, their formulations, and methods for using the formulations to treat diseases, disorders, and conditions treatable by the administration of TPGS-based formulations (e.g., Tocosol®, Sonus Pharmaceuticals paclitaxel/tocopherol/TPGS emulsion formulation) with a therapeutic drug suitably formulated for intravenous administration. In one embodiment, the therapeutic agent is paclitaxel.

Identification of Characteristics Defining Clinically Successful Nanoparticle Paclitaxel Formulations

The following provides an identification of characteristics defining advantageous activity of nanoparticle paclitaxel formulations. These defining characteristics are based on a comparison of IG-001 and nab-paclitaxel, each an effective and clinically successful nanoparticle paclitaxel formulation.

Methods.

Preclinical and clinical pharmacokinetic (PK) data of the formulations were compared to define the characteristics of an effective nanoparticle paclitaxel. The pharmacokinetic parameters were estimated by using the noncompartmental open model and the WinNonlin program (Pharsight Corp., Mountain View, Calif.). Descriptive statistics were computed for PK assessment. Regression analysis of AUCinf vs. dose was performed to gain an appreciation of dose proportionality. Tumor xenografts studies were also examined. Tumor inhibition relative to Taxol® was used to normalize across tumor types.

Designations.

The following designations are used for the formulations described herein.

Taxol® is an approved paclitaxel formulation comprising paclitaxel in a Cremaphor vehicle.

Abraxane® (nab-paclitaxel) is an albumin bound nanoparticle formulation marketed by Celgene against multiple indications (metastatic breast cancer and NSCLC). Recently, Celgene reported positive Phase III data against advanced pancreatic cancer as well as melanoma. Abraxane is cremophor-free with the following advantages over Taxol®: reduction of hypersensitivity reactions with elimination of steroid and histamine blocker premedication; infusion time of 30 min.; and an active transport by the albumin mediated transport pathway.

IG-001 is a chemical, polymer-bound nanoparticle paclitaxel formulation (a Cremophor-free, polymeric micelle formulation) that is approved in Korea and marketed by Samyang Biopharmaceuticals as Genexol-PM®. IG-001 is free of Cremophor-induced toxicities such as hypersensitivity reactions, prolonged or irreversible peripheral neuropathy, as well as altered lipoprotein patterns. IG-001 utilizes biodegradable diblock copolymer comprising methoxy poly(ethylene glycol)-poly(lactide) to form nanoparticles with paclitaxel (i.e., a hydrophobic core and a hydrophilic shell). IG-001 has a mean diameter of 25 nm with relatively low light scattering potential.

IG-002 is a cremophor-free, vitamin E-based paclitaxel emulsion incorporating a P-glycoprotein (Pgp) inhibitor. IG-002 is a particle size-based tumor targeting. IG-002 was developed to overcome a number of the limitations of the commercially available formulation of paclitaxel (Taxol®). Potential advantages as a result of elimination of the cremophor/ethanol delivery vehicle include the reduction of hypersensitivity reactions with reduction or elimination of steroid and histamine blocker premedications, the ability to bolus dose the emulsion in 15 minutes or less, and passive tumor targeting as a result of 200 nm emulsion particles being preferentially deposited in the tumor by the Enhanced Permeability and Retention (EPR) effect. The particle includes an inner core comprising a lipophilic material (α-tocopherol). At the interface between the lipophilic emulsion particle and its aqueous environment, a number of surfactants are employed, including the p-glycoprotein (pgp) inhibitor α-tocopherol polyethylene glycol succinate (TPGS). The surfactants, along with the manufacturing conditions, define and stabilize the emulsion particle size. IG-002 clinical development was halted by Sonus Pharmaceuticals due to failure in a Phase III clinical trial. IG-002, methods for making IG-002, and methods for using IG-002 are described in U.S. Pat. Nos. 6,458,373, 6,667,048, 6,727,280, 6,982,282, and 7,030155, each expressly incorporated herein by reference in its entirety.

Taxotere® is an approved docetaxel derivative.

Clinical Effectiveness.

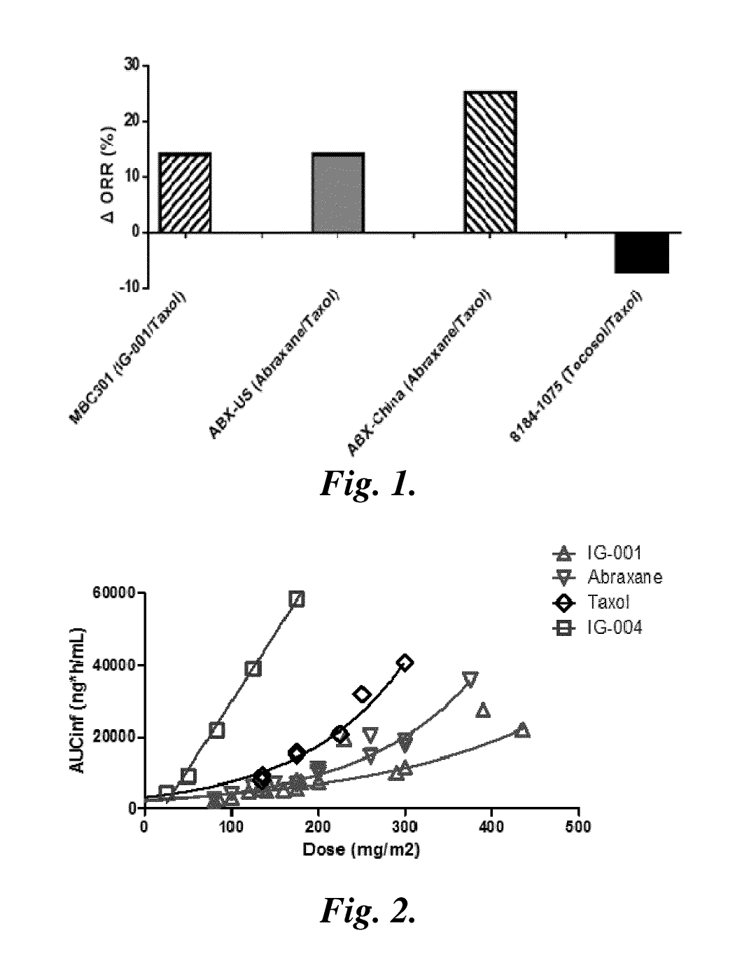

A Phase III, multicenter, randomized comparison of the safety and efficacy of weekly TOCOSOL® Paclitaxel (100 mg/m²) vs. weekly Paclitaxel Injection (80 mg/m²) in the treatment of metastatic breast cancer (MBC) was conducted. The primary endpoint was to compare the objective response rates (ORR) as assessed by RECIST in patients with MBC treated with weekly TOCOSOL® Paclitaxel or weekly Taxol as first-line or second-line therapy. MBC patients (1050) were screened and a total of 821 were randomized to receive either IG-002 (100 mg/m2 weekly, IV) or Crem-Pac (80 mg/m² weekly, IV) until disease progression. IG-001 Phase 3 trial: Treatment: IV infusion, 3 hrs, q3w, 6 cycles; N=212 (106 per arm) with IG-001 at 300 mg/m² and Paclitaxel (Taxol) at 175 mg/m². Objectives: Primary, ORR; and Secondary, OS, PFS, TTP, Duration of Overall Response. Abraxane Phase 3 trial (US): MBC patients were randomly assigned to 3-week cycles of either ABI-007 260 mg/m² intravenously without premedication (n=229) or standard paclitaxel 175 mg/m² intravenously with premedication (n=225) (Gradishar et al., 2005, JCO 23: 7794-7803). Abraxane Phase 3 trial (China): open-label, multicenter study, 210 patients with MBC were randomly assigned to receive Abraxane 260 mg/m² intravenously (i.v.) over 30 min every 3 weeks (q3w) with no premedication or Taxol 175 mg/m² i.v. over 3 h q3w with standard premedication (Guan et al., 2009, Asia-Pacific Journal of Clinical Oncology 5:165-174.). Both IG-001 and Abraxane demonstrated improved response rate versus Taxol. However, the stable nanoparticle formulation—IG-002/Tocosol—did not (see FIG. 1).

Tumor/Plasma Ratio.

IG-001 (Genexol-PM) is a Cremophor-free, polymeric micelle formulation of paclitaxel. The principle of polymeric micelles can be applied to both chemical polymer (IG-001) and biological polymer (Abraxane). Polymeric micelles span the spectrum of being stable in vivo (NK105 and IG-002) or unstable in vivo (Abraxane and IG-001). Therefore, the tumor plasma ratio of the various formulations was examined. However, if was found that tumor plasma ratio was unable to distinguish the clinically successful Abraxane and IG-001 from the clinically unsuccessful IG-002.

Clinical Pharmacokinetics.

The pharmacokinetics (PKs) of Abraxane, IG-001, Taxol and IG-002 were examined. The results are presented in FIG. 2 (expanded dose—proportionality for IG-001). Referring to FIG. 2, Abraxane PK deviated from proportionality above 300 mg/m², whereas IG-001 PK remained dose-proportional up to the highest dose of 435 mg/m².

IG-002 has higher plasma AUC than Taxol, whereas IG-001 and Abraxane have lower plasma AUC than Taxol.

IG-001 range for PK dose-proportionality is the most expanded of the four paclitaxel formulations examined.

The dose proportionality of Abraxane and IG-001 clearly separated these formulations from Taxol and IG-002.

Paclitaxel Release.

The release of paclitaxel from an unstable nanoparticle (Abraxane) versus a stable nanoparticle (IG-002) was examined. Paclitaxel release from formulation was tested using equilibrium dialysis. Briefly, paclitaxel, IG-002, Taxol or reconstituted Abraxane was added to one side of the well and blank buffer to the other side. Sample was taken from the buffer side for the analysis of the appearance of free paclitaxel.

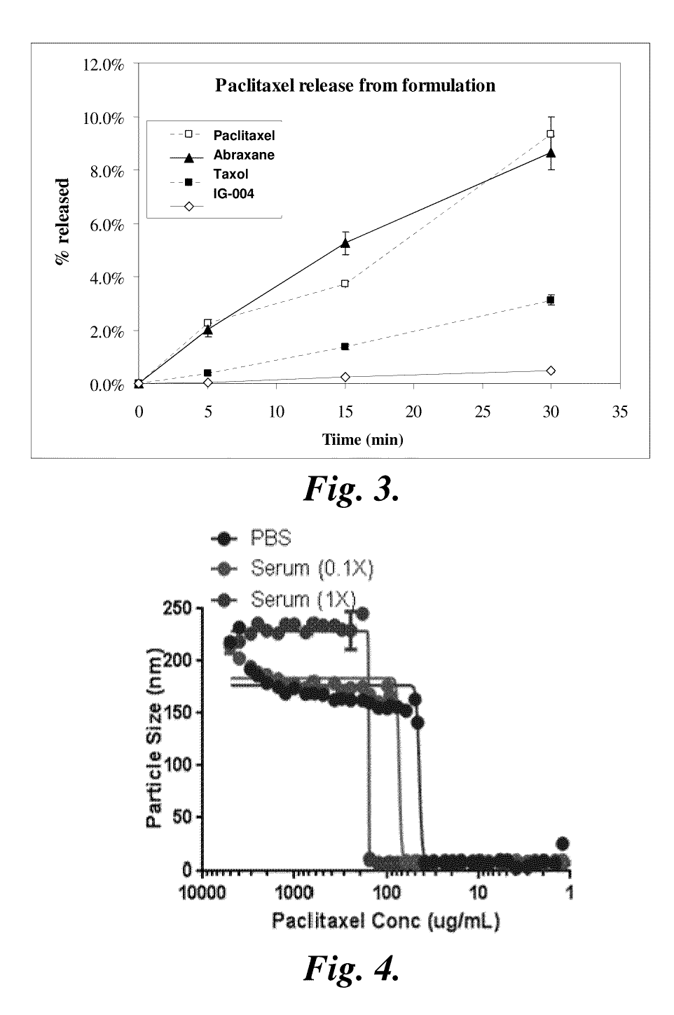

FIG. 3 shows that drug release appears to be linear over 30 minutes following addition of paclitaxel in organic solvent (neat paclitaxel), IG-002, Taxol, or Abraxane to the donor side at a nominal concentration of 10 μg/mL paclitaxel. The drug release profile from Abraxane appears similar to neat paclitaxel. Drug release is slowest for IG-002 (0.5% at 30 minutes, statistically significant versus the other three groups).

Stable nanoparticle is associated with slow release of paclitaxel. The rapid release of paclitaxel from unstable nanoparticle such as Abraxane is probably responsible for its effective use of albumin mediated transport.

Paclitaxel release separated the clinically successful formulation (Abraxane) from IG-002.

In Vivo Particle Size of Abraxane/IG-001 (Genexol-PM).

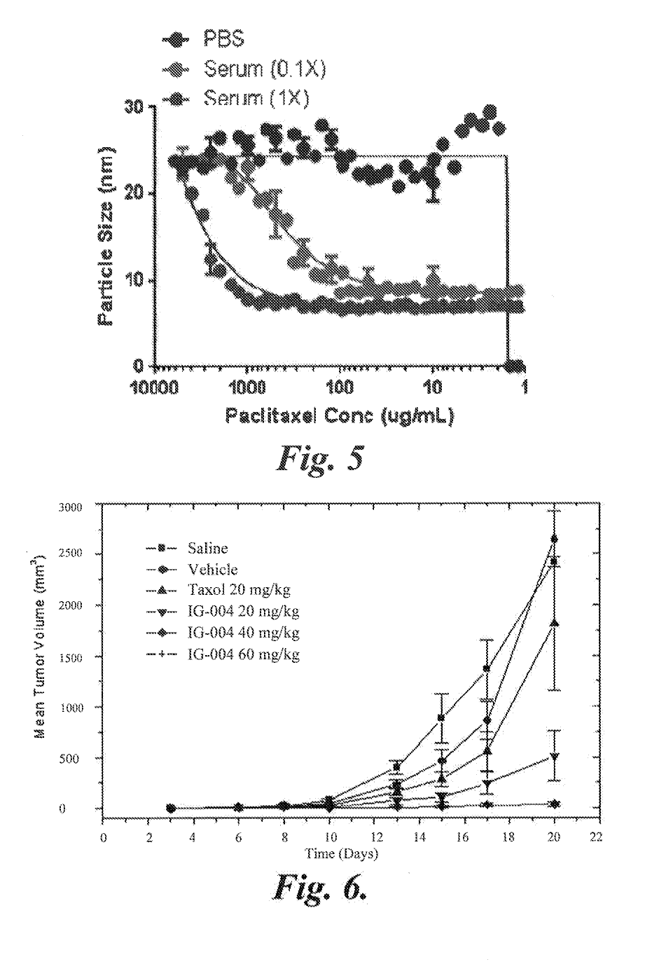

IG-001 (Genexol-PM) is a Cremophor-free, polymeric micelle formulation of paclitaxel utilizing biodegradable di-block copolymer composed of methoxy poly(ethylene glycol)-poly(lactide) to form nanoparticles with paclitaxel containing a hydrophobic core and a hydrophilic shell. IG-001 has a mean diameter of 25 nm with relatively low light scattering potential. Stability of the nanoparticle was examined across various concentrations to determine the approximate critical micelle concentration (CMC). IG-001 rapidly dissociates from intact nanoparticles upon dilution in serum at concentrations less than 50 ug/ml—higher than the Cmax of IG-001—following a 3 hr infusion (FIG. 5). The CMC is higher than experimental maximum drug level. Therefore, once administered, IG-001 readily gives up its paclitaxel cargo to endogenous drug transporters for transport into the underlying tissues. Similar phenomenon was observed for Abraxane. The data suggest that particle size need to be small to be effective, at least smaller than Tocosol and implying that active transport of the drug out of circulation into underlying tissue is the primary reason for clinical effectiveness.

Tumor Xenograft.

The antitumor activities of stable (IG-002) and unstable nanoparticles (IG-001) compared to Taxol were examined.

IG-002 B16 study. Murine B16 melanoma tumor model—female B6D2F mice were subcutaneously implanted with 10⁶ B16 melanoma tumor cells. Saline, IG-004-vehicle, IG-002, or Taxol were administered intravenously on a schedule of either q3dx5 or q4dx5.

The results are shown in FIG. 6. At equal dose of 20 mg/kg, IG-002 was more effective than Taxol.

IG-001 B16 study. B16 melanoma cells (106 cells in volume of 200 uL) were innoculated into the flank of female C57Bl/6 mice. IG-001-vehicle, IG-001, Taxol vehicle, and Taxol were dosed at Q1dx3.

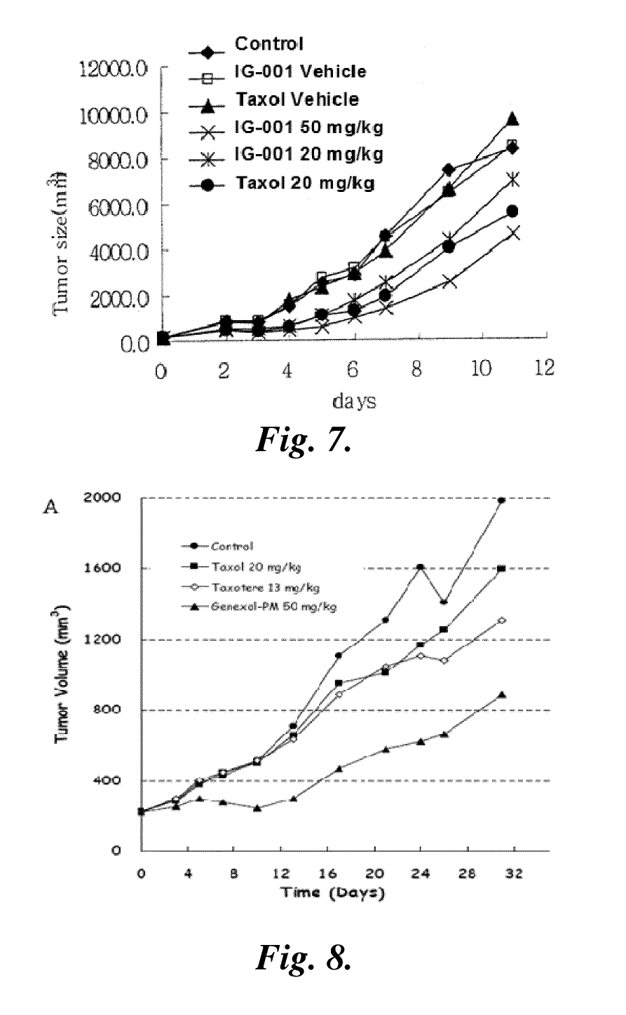

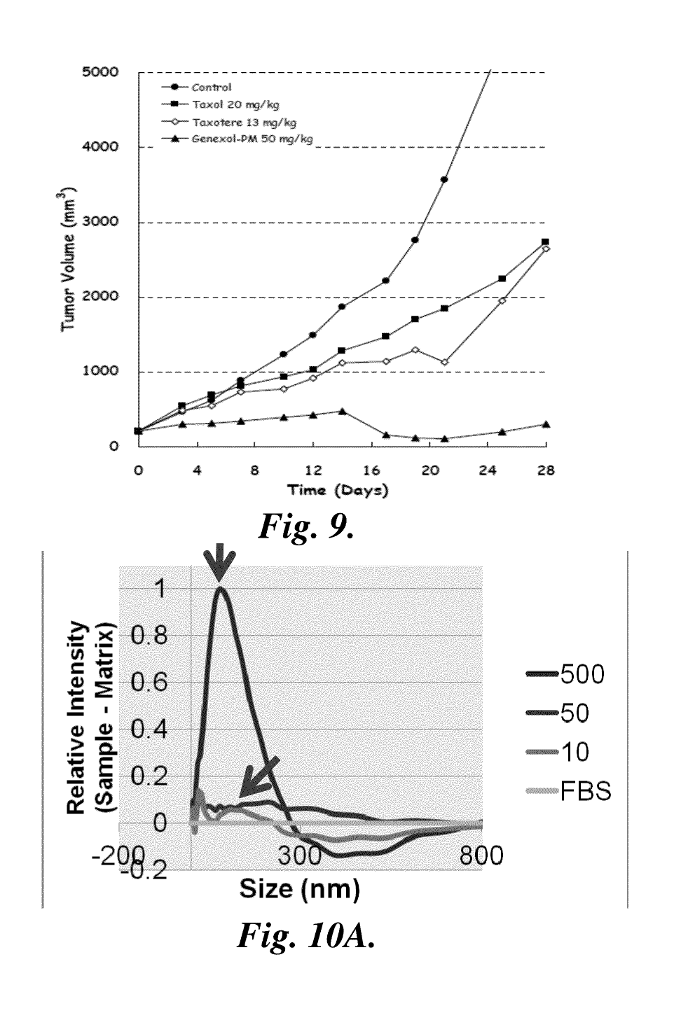

The results are shown in FIG. 7. At equal dose of 20 mg/kg, IG-001 was similar to Taxol. Similar findings have been reported for Abraxane versus Taxol.

IG-001 DLD-1 and NCI-H1299 studies. IG-001 (Genexol-PM), Taxol, Taxotere, and saline were administered intravenously at schedule of Q4dx3.

The results are shown in FIGS. 8 (DLD-1) and 9 (NCI-H1299). At equitoxic dose IG-001 was superior to Taxol (this is true across all xenograft examined including the pancreatic xenografts). Similar findings been reported for Abraxane.

Plasma Instability.

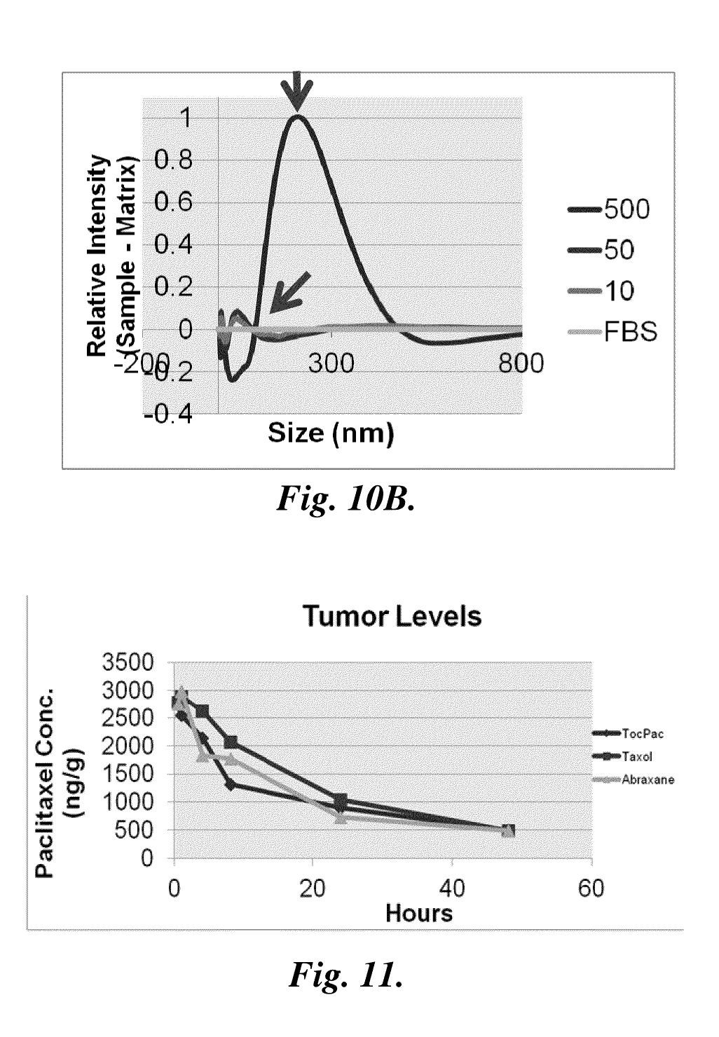

Because both IG-001 and Abraxane are clinically successful, their stability in plasma (an extension of the paclitaxel release assay) was examined. Stability of the nanoparticle was examined across various concentrations to determine the approximate critical micelle concentration (CMC). FIG. 10A illustrates IG-001 nanoparticles at concentrations above its critical micelle concentration (CMC) (see peak) and the absence of nanoparticles at physiological concentration (see second arrow). FIG. 10B illustrates Abraxane nanoparticles at concentrations above its critical micelle concentration (CMC) (see peak) and the absence of nanoparticles at physiological concentration (see second arrow).

As shown in FIGS. 10A and 10B, Abraxane and IG-001 have similar CMCs (higher than expected Cmax of the two formulations). Therefore, during administration to the patient the two formulations should readily release their drug load for transport by the albumin drug transport pathway.

Tumor Distribution.

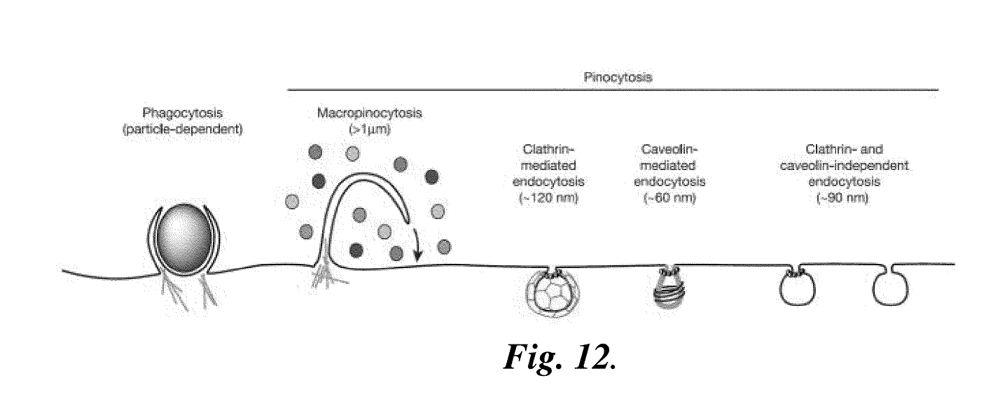

Paclitaxel tumor distribution for IG-002, Taxol, and Abraxane were examined in a MDA-MB-435 xenograft model. The results are shown in FIG. 11. Tumor accumulation of paclitaxel was similar for all three formulations at dose level of 10 mg/kg. Therefore, the conclusion is that tumor accumulation in mice does not predict clinical success. This result suggests that the enhanced permeability and retention (EPR) effect, which IG-002 was engineered to exploit, may not be active in humans.

Results.

The defining characteristics of nab-paclitaxel are its rapid tissue penetration such that tumor/plasma drug ratio was greater than Taxol, with 1.9-fold advantage for nab-paclitaxel. Formulations with tumor accumulation greater than Taxol without corresponding high tumor/plasma ratio were ineffective. IG-001 and nab-paclitaxel exhibited the similar profiles: high tissue penetration, high tumor/plasma ratio, dose proportional PK in human, higher maximum tolerated dose (MTD) in tumor xenograft studies, higher MTD during Phase I dose escalation study in human. Correspondingly, IG-001 and nab-paclitaxel have similar activities in tumor xenografts models (e.g., higher MTD and better efficacy at equitoxic dose versus Taxol). More importantly, IG-001 exhibited activity against poorly perfused pancreatic xenografts (MIAPaCa-2, PANC-1).

The best predictors of successful clinical outcome are clinical PK and possibly paclitaxel release. Tumor/plasma ratio and xenograft studies were not predictive suggesting that tumor accumulation of nanoparticle in mice could be different from tumor accumulation in human. Through all examinations, IG-001 was similar to Abraxane.

Conclusions.

A non-albumin-based paclitaxel formulation (IG-001) was found to have similar properties to the albumin-based paclitaxel formulation (nab-paclitaxel). The clinical development of IG-001 may provide the next generation paclitaxel nanoparticle formulation that can be more readily modified than nab-paclitaxel (Abraxane). More importantly, this model could be used to develop other nanoparticle drugs against difficult to perfuse tumors such as pancreatic cancers.

Nanoparticle Formulations and Active Transport Mechanisms for Therapeutic Agent Delivery

It was surprising that a stable nanoparticle (IG-002, Tocosol®) made to take advantage of the EPR effect and utilizing passive transport in mouse models was not found effective in human clinic trials. The present invention addresses this failure and provides for the construction of stable nanoparticle formulations targeted to active transport mechanisms. Realizing that the limitation on size on whether the nanoparticle can be transported across the endothelial barrier, the present invention ties the size limitation to the targeting agent. The nanoparticle cannot be larger than the size of the transport organelles being exploited. As shown in FIG. 12, clathrin-mediated pathways require a nanoparticle size less than about 120 nm, caveolin-mediated pathways require a nanoparticle size less than about 60 nm, and clathrin and caveolin independent pathways require a nanoparticle size less than about 90 nm.

The nanoparticle therapeutic agents of the invention utilize active transport mechanisms for entry into the cell of interest. As used herein, the term “active transport” refers a transcytosis process whereby the nanoparticle of the invention is transported into a cell of interest or from one part of a cell to another. In the practice of the invention, transcytosis processes include caveolae-mediated transcytosis and clatherin-mediated transcytosis. In caveolae-mediated transcytosis, the caveola are used as the transporter. The major structural component of caveola is caveolin. In clathrin-mediated transcytosis, clathrin forms the structure of the transport vesicle.

Nanoparticle Therapeutic Agent Formulations

In one aspect, the invention provides a nanoparticle therapeutic agent comprising a therapeutic agent in a carrier (or vehicle) (e.g., an emulsion nanoparticle or a polymeric micelle nanoparticle). The nanoparticle of the invention has a size sufficient to be taken into and/or across a cell of interest by active transport through a cell surface organelle. Active transport includes transcytosis, particularly caveolae-mediated transcytosis, clathrin-mediated transcytosis, and clathrin- and caveolae-independent transcytosis. For these process, the nanoparticle size is adapted to suit these transcytosis mechanisms (e.g., 60 nm, 120 nm, and 90 nm, respectively).

The nature of the therapeutic agent delivered by the nanoparticle of the invention is not particularly critical. Representative therapeutic agents are described below.

In certain embodiments, the nanoparticle further comprises a targeting agent to selectively target the nanoparticle to a cell or cells of interest. Representative targeting agents are described below.

In other embodiments, the nanoparticle is a theranostic and further includes an imaging agent. Representative imaging agents are described below.

In further embodiments, the nanoparticle includes both a targeting agent and an imaging agent.

In one embodiment, the nanoparticle comprises:

(a) a core comprising a therapeutic agent and a tocopherol; and

(b) a shell comprising a tocopherol polyethylene glycol derivative,

wherein the shell surrounds the core, and

wherein the nanoparticle has a size sufficient to be taken into and/or across a human cell by active transport through a cell surface organelle.

The nanoparticles of the invention deliver therapeutic agents. Therapeutic agents effectively delivered by the nanoparticles of the invention include small organic molecules, peptides, aptamers, proteins, and nucleic acids. In certain embodiments, the therapeutic agent is a small organic molecule such as a difficulty water-soluble small molecule.

Suitable therapeutic agents include conventional therapeutic agents, such as small molecules; biotherapeutic agents, such as peptides, proteins, and nucleic acids (e.g., DNA, RNA, cDNA, siRNA); and cytotoxic agents, such as alkylating agents, purine antagonists, pyrimidine antagonists, plant alkaloids, intercalating antibiotics, antitumor antibiotics (e.g., trastuzumab), binding epidermal growth factor receptors (tyrosine-kinase inhibitors), aromatase inhibitors, anti-metabolites (e.g., folic acid analogs, methotrexate, 5-fluoruracil), mitotic inhibitors (e.g., a taxane, taxine or taxoid such as taxol, paclitaxel, docetaxel), growth factor inhibitors, cell cycle inhibitors, enzymes, topoisomerase inhibitors, biological response modifiers, anti-hormones, anti-androgens, and various cytokines for immunotherapy. Representative cytotoxic agents include BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminoglutethimide, anastrozole, amsacrine, asparaginase, mitoxantrone, mitotane, and amifostine. Suitable therapeutic drugs include siRNAs and antitumor tumor drugs than function in cytoplasma.

In certain embodiments, the therapeutic agent is a taxane, taxine, or taxoid. In certain embodiments, the therapeutic agent is paclitaxel, which is a taxane that has found success in human clinical trials for treating tumors.

Paclitaxel is a member of the taxane dipterine family. Paclitaxel has a molecular formula of C₄₇H₅₁NO₁₄ and a molecular weight of 853.93. Paclitaxel can be prepared by extraction from the bark and needles of the Yew tree (Taxus yunnanensis). Alternatively, paclitaxel is prepared synthetically or semi-synthetically. Some embodiments include paclitaxel derivatives, for example benzoate derivatives of paclitaxel such as 2-debenzoyl-2-aroyl and C-2-acetoxy-C-4-benzoate paclitaxel, 7-deoxytaxol, C-4 aziridine paclitaxel, as well as various paclitaxel conjugates with natural and synthetic polymers, particularly with fatty acids, phospholipids, and glycerides and 1,2-diacyloxypropane-3-amine. As used herein, the term “paclitaxel” refers to paclitaxel, a paclitaxel derivative, or a paclitaxel analog.

Other members of the family of related molecules called taxoids, taxanes, or taxines are also within the scope of the present invention. The taxane can be any anti-mitotic taxane, taxane derivative or analog. It is generally believed that the mechanism of action of taxanes involves promoting formation and hyperstabilization of microtubules, thus blocking cell division. As used herein, the term “taxane” refers to a taxanes, taxines, and taxoids, as well as derivatives or analogs thereof.

In some embodiments, the taxane, taxane derivative, or taxane analog can include, for example, docetaxel (Taxotere®, Aventis Pharmaceuticals); spicatin; taxane-2,13-dione, 5β,9β,10β-trihydroxy-, cyclic 9,10-acetal with acetone, acetate; taxane-2,13-dione, 5β,9β,10β-trihydroxy-, cyclic 9,10-acetal with acetone; taxane-2β,5β,9β,10β-tetrol, cyclic 9,10-acetal with acetone; taxane; cephalomannine-7-xyloside; 7-epi-10-deacetylcephalomannine; 10-deacetylcephalomannine; cephalomannine; taxol B; 13-(2′,3′-dihydroxy-3′ phenylpropionyl)baccatin III; yunnanxol; 7-(4-azidobenzoyl)baccatin III; N-debenzoyltaxol A; O-acetylbaccatin IV; 7-(triethylsilyl)baccatin III; 7,10-di-O-[(2,2,2,-trichloroethoxy)carbonyl]baccatin III; baccatin III 13-O-acetate; baccatin diacetate; baccatin; baccatin VII; baccatin VI; baccatin IV; 7-epi-baccatin III; baccatin V; baccatin I; baccatin III; baccatin A; 10-deactyl-7-epitaxol; epitaxol; 10-deacetyltaxol C; 7-xylosyl-10-deacetyltaxol; 10-deacetyltaxol-7-xyloside; 7-epi-10-deacetyltaxol; 10-deactyltaxol; or 10-deactyltaxol B, as well as any combination of two or more of the foregoing molecules.

In certain embodiments of the invention that utilize the nanoparticle as an emulsion droplet, the nanoparticle includes a tocopherol. Tocopherols are a family of natural and synthetic compounds, also known by the generic names tocols or vitamin E. Among the tocopherols, α-tocopherol is the most abundant and active form of this class of compounds. Other members of this class include α-, β-, γ-, and δ-tocotrienols, and α-tocopherol and derivatives such as tocopherol acetate, phosphate, succinate, nicotinate and linoleate. As used herein, the term “tocopherol” refers to any member of the tocopherol family. In certain embodiments, the tocopherol is α-tocopherol.

In embodiments of the invention that utilize the nanoparticle as an emulsion droplet and a tocopherol, the nanoparticle may also include a tocopherol polyethylene glycol derivative. The tocopherol polyethylene glycol derivative is an ester or ether of a tocopherol acid and polyethylene glycol. In one embodiment, the tocopherol polyethylene glycol derivative is a tocopherol polyethylene glycol succinate (TPGS). A representative TPGS is d-α-tocopherol polyethylene glycol 1000 succinate (MW=˜1513). TPGS is a vitamin E derivative in which polyethylene glycol subunits are attached by a succinic acid ester at the ring hydroxyl of the vitamin E molecule. TPGS is a non-ionic surfactant (HLB=16-18). Various chemical derivatives of vitamin E TPGS including ester and ether linkages of various chemical moieties are included within the definition of vitamin E TPGS. TPGS is reported to inhibit P-glycoprotein, a protein that contributes to the development of multi-drug resistance. In some embodiments, the diester content of TPGS in the formulations of the invention does not exceed 20%, and the free polyethylene glycol does not exceed 10% (w/w).

In certain embodiments, the ratio of tocopherol to tocopherol polyethylene glycol derivative is from about 1:1 to about 10:1 w/w.

In certain embodiments, the ratio of therapeutic agent to the tocopherol is from about 0.2:1 to about 0.4:1 w/w.

In certain embodiments, the nanoparticle further comprises a polyethylene glycol. “Polyethylene glycol” (PEG) is a hydrophilic, polymerized form of ethylene glycol, consisting of repeating units of the chemical structure: (—CH₂—CH₂—O—). The general formula for polyethylene glycol is H(OCH₂CH₂)ₙOH. The molecular weight ranges from 200 to 10,000. Such various forms are described as PEG-200, PEG-400, and the like. In a preferred embodiment, the therapeutic agents of the compositions of the invention can initially be solubilized in non-volatile co-solvents such as dimethylsulfoxide (DMSO), dimethylamide (DMA), propylene glycol (PG), polyethylene glycol (PEG), N-methyl-2-pyrrolidone (NMP) and polyvinylpyrrolidone (PVP); NMP or a water-soluble polymer such as PEG or PVP are particularly preferred.

A major advantage/improvement of using PEG-400 to solubilize therapeutic agents rather than alcohols such as ethanol is that a volatile solvent does not have to be removed or diluted prior to administration of the therapeutic agent. The final polyethylene glycol levels in the emulsion can be varied from about 1 to about 50% (w/w), for example from about 1 to about 25%, or from about and more preferably from about 1 to about 10%. Suitable polyethylene glycol solvents are those with an average molecular weight between 200 and 600, preferably 300 and 400. In the case of self-emulsified systems for oral administration, high molecular weight PEGs (1,000-10,000) can also be included as solidification agents to form semi-solid formulations which can be filled into hard gelatin capsules.

In other embodiments, the nanoparticle further comprises a polyoxypropylene-polyoxyethylene glycol nonionic block copolymer. Representative polyoxypropylene-polyoxyethylene glycol nonionic block copolymers include “poloxamers” or “pluronics,” which are synthetic block copolymers of ethylene oxide and propylene oxide having the general structure: H(OCH₂CH₂)ₐ(OCH₂CH₂CH₂)_{b}(OCH₂CH₂)ₐOH. The following variants based on the values of a and b are commercially available from BASF Performance Chemicals (Parsippany, N.J.) under the trade name Pluronic and which consist of the group of surfactants designated by the CTFA name of poloxamer 108, 188, 217, 237, 238, 288, 338, 407, 101, 105, 122, 123, 124, 181, 182, 183, 184, 212, 231, 282, 331, 401, 402, 185, 215, 234, 235, 284, 333, 334, 335, and 403. For the most commonly used poloxamers 124, 188, 237, 338, and 407 the values of a and b are 12/20, 79/28, 64/37, 141/44 and 101/56, respectively.

In further embodiments, the nanoparticle comprises a polyethylene glycol and a polyoxypropylene-polyoxyethylene glycol nonionic block copolymer.

In certain embodiments, the nanoparticles of the invention are formulated as emulsions, microemulsions, or polymeric micelles.

The term “emulsion” refers to a colloidal dispersion of two immiscible liquids in the form of droplets, whose diameter, in general, are between 0.1 and 3.0 microns and which is typically optically opaque, unless the dispersed and continuous phases are refractive index matched. Such systems possess a finite stability, generally defined by the application or relevant reference system, which may be enhanced by the addition of amphiphilic molecules or viscosity enhancers.

The term “microemulsion” refers to a thermodynamically stable isotropically clear dispersion of two immiscible liquids, such as oil and water, stabilized by an interfacial film of surfactant molecules. The microemulsion has a mean droplet diameter of less than 200 nm, in general between 10-50 nm. In the absence of water, mixtures of oil(s) and non-ionic surfactant(s) form clear and isotropic solutions that are known as self-emulsifying drug delivery systems (SEDDS) and have successfully been used to improve lipophilic drug dissolution and oral absorption.

The term “polymeric micelle” refers to a micellular nanoparticle (core/shell: shell comprising amphiphilic compounds such as amphiphilic polymers). In the practice of the invention, the polymeric micelle has mean particle diameter of less than 200 nm, in general between 10-50 nm. Polymeric micelles of the invention can be used to improve lipophilic drug dissolution and oral absorption.

Representative polymeric micelles of the invention include mPEG-pAsp micelles having a particle size as described herein for nanoparticles of the invention, which can be provided in accordance with the methods of the invention. Representative mPEG-pAsp micelles that can be transformed into polymeric micelles of the invention include those that deliver paclitaxel (NK105), doxorubicin (N911), and SN38 (NK012). In certain embodiments, polymeric micelles of the invention include diblock amphipathic polymers. Suitable diblock amphiphilic polymers include mPEG-PDLLA, mPEG-PLGA, mPEG-pAsp, and PVP-b-PNIPAM.

In certain embodiments of the invention, the nanoparticle includes a therapeutic agent (e.g., paclitaxel), a tocopherol (e.g., α-tocopherol), and a tocopherol polyethylene glycol derivative (e.g., tocopherol polyethylene glycol succinate).

In other embodiments, in addition to a therapeutic agent, a tocopherol, and a tocopherol polyethylene glycol derivative, the nanoparticle further includes a polyethylene glycol and a polyoxypropylene-polyoxyethylene glycol nonionic block copolymer.

In one particular embodiment, the nanoparticle comprises paclitaxel, α-tocopherol, the tocopherol polyethylene glycol succinate, a polyethylene glycol, and a polyoxypropylene-polyoxyethylene glycol nonionic block copolymer.

For embodiments of the invention in which the nanoparticle is an emulsion droplet (i.e., an oil phase dispersed as droplets in an aqueous phase), the size of the nanoparticle can be controlled by, for example, lowering the therapeutic agent load. For example, lowering the paclitaxel load in a paclitaxel/tocopherol/TPGS-containing nanoparticle will allow for a reduction in nanoparticle size thereby advantageously rendering the nanoparticle readily available for uptake into cells of interest by active transport (transcytosis). The reduction of particle size to that effective for transcytosis will also have the overall effect of reducing plasma accumulation. Alternatively, nanoparticle size can be reduced by mechanical means during emulsion formation by, for example, variation of shearing conditions.

As noted above, in certain embodiments, the nanoparticle of the invention further includes a targeting agent. Suitable targeting agents include compounds and molecules that direct the nanoparticle to the site of interest. Suitable targeting agents include tumor targeting agents. Representative targeting agents include small molecules, peptides, proteins, aptamers, and nucleic acids. Representative small molecule targeting agents include folic acid, methotrexate, non-peptidic RGD mimetics, vitamins, and hormones. Representative peptide targeting agents include RGD (avβ3 integrin), chlorotoxin (MMP2), and VHPNKK (endothelial vascular adhesion molecules). Representative protein targeting agents include antibodies against the surface receptors of tumor cells, such as monoclonal antibody A7 (colorectal carcinoma), herceptin (Her2/ner), rituxan (CD20 antigen), and ligands such as annexin V (phosphatidylserine) and transferrin (transferrin receptor). Representative aptamer targeting agents include A10 RNA aptamer (prostate-specific membrane antigen) and Thrm-A and Thrm-B DNA aptamers (human alpha-thrombin protein). Targets for the agents noted above are in parentheses. Representative nucleic acid targeting agents include DNAs (e.g., cDNA) and RNAs (e.g., siRNA). In certain embodiments, the targeting agent is an antibody or functional fragment thereof. The targeting agent can be covalently coupled to a component of the nanoparticle (e.g., tocopherol polyethylene glycol derivative, TPGS).

In certain embodiments, the nanoparticle of the invention further includes an imaging agent. Suitable imaging agents include magnetic resonance imaging agents, fluorescent agents, ultrasound imaging agents, radiolabels, surface plasmon resonance imaging agents. Representative magnetic resonance imaging agents include iron- (e.g., iron oxide) and gadolinium-based agents. Representative fluorescent agents include fluorescent agents that emit visible and near-infrared light (e.g., fluorescein and cyanine derivatives). Representative fluorescent agents include fluorescein, OREGON GREEN 488, ALEXA FLUOR 555, ALEXA FLUOR 647, ALEXA FLUOR 680, Cy5, Cy5.5, and Cy7. Representative ultrasound imaging agents include carbon- and metal-based agents. Representative radiolabels includes ¹³¹I for radioimaging and ⁶⁴Cu, ¹⁸F, and ¹¹C for positron emission tomography (PET). Representative surface plasmon resonance imaging agents include gold-based agents. The imaging agent can be covalently coupled to a component of the nanoparticle (e.g., tocopherol polyethylene glycol derivative, TPGS).

In certain embodiments, the nanoparticle includes one or more therapeutic agents and one or more targeting agents.

In other embodiments, the nanoparticle includes one or more therapeutic agents and one or more imaging agents.

In further embodiments, the nanoparticle includes one or more therapeutic agents, one or more targeting agents, and one or more imaging agents.

In another aspect of the invention, an emulsion is provided. In one embodiment, the emulsion comprises a water phase; and an oil phase comprising a plurality of nanoparticles of the invention. In certain embodiments, the therapeutic agent is present in amount from about 1 to about 20 mg/mL. In other embodiments, the therapeutic agent is present in amount from about 5 to about 10 mg/mL. In further embodiments, the therapeutic agent is present in amount from about 2 to about 5 mg/mL.

In a further aspect, the invention provides method for treating a disease, condition, or disorder treatable by administering a specific therapeutic agent (i.e., an agent that is known to be effective for the treating a particular disease, condition, or disorder). In one embodiment, the method includes administering a therapeutically effective amount of a nanoparticle of the invention, or an emulsion or formulation of the invention comprising the specific therapeutic agent, to a subject in need thereof.

In certain embodiments, the therapeutic agent is taken into and/or across a targeted cell by active transport through a cell surface organelle. Representative active transport pathways include caveolin-mediated endocytosis, clathrin-mediated endocytosis, and caveolin- and clathrin-independent endocytosis.

In certain embodiments, the therapeutic agent is a taxane (e.g., paclitaxel) and the disease, condition, or disorder treatable by administering a taxane is a cancer. In certain embodiments, therapeutic agent is paclitaxel. Cancers treatable by administration of paclitaxel and the nanoparticles and formulation of the invention include pancreatic, ovarian, bladder, lung, and breast cancer.

Nanoparticle Conjugates

In another aspect of the invention, a nanoparticle targeting agent conjugate is provided. In one embodiment, the nanoparticle comprises a core comprising a therapeutic agent and a tocopherol; a shell comprising a tocopherol polyethylene glycol derivative, wherein the shell surrounds the core; and a targeting agent.

In an embodiment of this aspect, the invention provides an emulsion comprising a water phase; and an oil phase comprising a plurality of the nanoparticle targeting agent conjugates of the invention.

In a further embodiment of this aspect, the invention provides a method for treating a disease, condition, or disorder treatable by administering a specific therapeutic agent, comprising administering to a subject in need thereof a therapeutically effective amount of the nanoparticle targeting agent conjugate of the invention or a formulation (e.g., emulsion) thereof comprising the specific therapeutic agent.

In a further aspect of the invention, a nanoparticle imaging agent conjugate is provided. In one embodiment, the nanoparticle comprises a core comprising a therapeutic agent and a tocopherol; a shell comprising a tocopherol polyethylene glycol derivative, wherein the shell surrounds the core; and an imaging agent.

In an embodiment of this aspect, the invention provides an emulsion comprising a water phase; and an oil phase comprising a plurality of the nanoparticle imaging agent conjugates of the invention.

In a further embodiment of this aspect, the invention provides a method for treating a disease, condition, or disorder treatable by administering a specific therapeutic agent, comprising administering to a subject in need thereof a therapeutically effective amount of the nanoparticle imaging agent conjugate of the invention or a formulation (e.g., emulsion) thereof comprising the specific therapeutic agent.

Nanoparticle conjugates that include a targeting agent and an imaging agent are also within the scope of the invention.

For the nanoparticle conjugates of the invention, the nanoparticle components, the targeting agents, imaging agents, emulsions, polymeric micelles, and methods of use are as described above for the nanoparticle therapeutic agent formulations.

Nanoparticle Platform: Polymeric Micelles Provide Stable Nanoparticles.

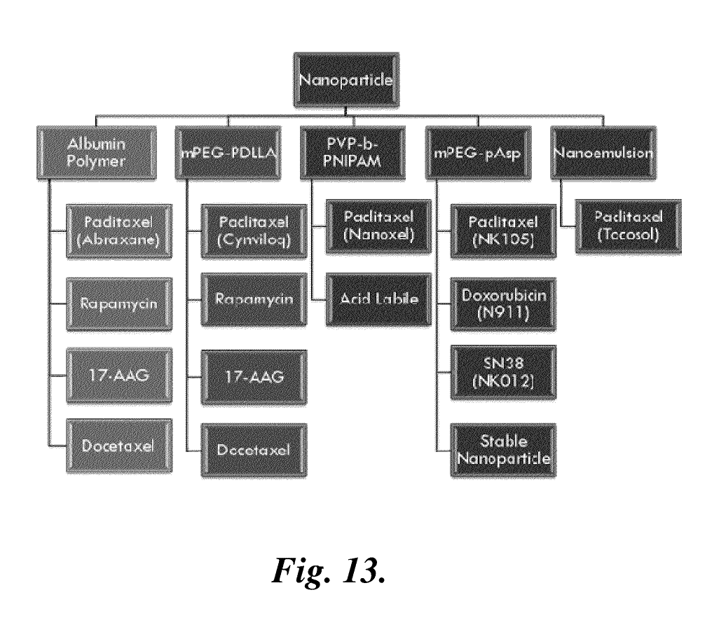

Polymeric micelles, such as IG-001, are inherently unstable in plasma/blood and give rise to smaller breakdown product. However, it is possible also to make small stable nanoparticles using the same technology as described herein. It will be appreciated that the present invention relates to any nanoparticle formulation (prepared, for example, by the methods of the invention) that provides a small nanoparticle that can be actively transported across the endothelium barrier to underlying tissues which result in blood exposure as shown by low AUC and Cmax. Examples of representative nanoparticle include those shown in FIG. 13 using mPEG-pAsp diblock polymer.

As used herein, the term “nanoparticle(s)” includes “emulsion nanoparticle(s)” and “polymeric micelle nanoparticle(s).”

Methods of Administration

In another aspect of the invention, methods for administering therapeutic agents are provided. In the methods, nanoparticles of the invention and formulations that include the nanoparticles are administered.

The term “therapeutically effective amount” refers to an optimized amount of taxane/tocopherol such that the desired antitumor activity is provided without significant side effects. The amount of a given drug that will be effective in the treatment of a particular tumor will depend in part on the severity of the tumor, and can be determined by standard clinical techniques. In addition, in vitro or in vivo assays may optionally be employed to help identify optimal dosage ranges. Effective doses may be extrapolated from dose-response curves derived from in vitro or animal model test systems. The precise dosage level should be determined by the attending physician or other health care provider and will depend upon well-known factors, including route of administration, and the age, body weight, sex and general health of the individual; the nature, severity and clinical stage of the tumor(s); and the use (or not) of concomitant therapies. Of course, the skilled person will realize that divided and partial doses are also within the scope of the invention. For example, it may be appropriate to administer a weekly dose of about 80 mg/m² as a twice weekly dose of about 40 mg/m².

The term “area-under-the-curve” or “AUC” refers to the integral of taxane concentration in a defined body compartment (e.g., blood, plasma or serum) over time, from zero to infinity or any interim time point. Thus, AUC₀₋ₜ is the non-extrapolated area under the concentration-time curve from time 0 to a defined time point t, and AUC_{0-∞} is the extrapolated area under the concentration-time curve from time 0 to infinity.

The term “antitumor activity” refers to the efficacy of a nanoparticle (e.g., taxane-containing) composition in providing a therapeutic benefit to a subjects suffering from a tumor. The responses to treatment in solid tumors are evaluated using guidelines such as those published by the World Health Organization in 1979 (WHO handbook for reporting results of cancer treatment (1979), World Health Organization Offset Publication No. 48); by Miller et al. in 1981 (Miller et al. (1981) Cancer 47:207-214); and the response evaluation criteria in solid tumors (RECIST) by Therasse et al. in 2000 (Therasse et al. (2000) J. Natl. Cancer Inst. 92:205-216). For example, according to the RECIST criteria, a complete response is defined as the disappearance of all target lesions, a partial response is defined as at least a 30% decrease in the sum of the longest diameter of target lesions, progressive disease is defined as at least a 20% increase in the sum of the longest diameter of target lesions or the appearance of new lesions, and stable disease is defined as neither sufficient shrinkage to qualify for partial response nor sufficient increase to qualify for progressive disease. Thus, a complete or a partial response and stable disease represent the presence of anti-tumor activity, and progressive disease represents the absence of anti-tumor activity. Other evidence of anti-tumor activity is provided by, for example, when the administration of taxane reduces the overall tumor burden, results in an objective response, slows tumor progression, prevents tumor recurrence, prevents the appearance of new tumor lesions, results in a partial or complete response in a tumor lesion, or results in a therapeutic benefit to the subject.

In one embodiment, the invention provides methods for administering a nanoparticle formulation comprising at least one tocopherol and at least one taxane. In certain embodiments, the taxane is paclitaxel. In some embodiments, the tocopherol is α-tocopherol. Some embodiments of the invention provide methods for administering paclitaxel in an oil-in-water emulsion with the following composition:

| Component | Amount per mL | |
|---|---|---|
| Paclitaxel | 5-20 | mg |
| d,1 α-Tocopherol (Vitamin E) | 20-100 | mg |
| d-α-Tocopherol polyethylene glycol 1000 | 2-100 | mg |
| succinate (TPGS) | | |
| Poloxamer 407 (Pluronic F127) | 5-20 | mg |
| Polyethylene glycol 400 (PEG 400) | 40-80 | mg |

| | |
|---|---|
| Water for Injection | q. s. |

In one embodiment, the emulsion comprises about 10 mg/mL paclitaxel, about 80 mg/mL tocopherol, about 50 mg/mL TPGS, about 10 mg/mL poloxamer 407, and about 60 mg/mL PEG 400. In some embodiments, the emulsion incorporates paclitaxel at a nominal concentration of about 10 mg/L. In some embodiments, the paclitaxel concentration is between about 6 mg/mL to about 10 mg/mL. In some embodiments, the taxane concentration is more than 10 mg/ml. Some embodiments of the invention provide methods for administering a ready-to-use formulations that requires no dilution or mixing with excipients or other carriers prior to administration.

In some embodiments, the dose of taxane administered is between about 15 and about 225 mg/m². Some embodiments provide for administration of a taxane at doses between about 25 and about 225 mg/m². Some embodiments provide for administration of a taxane at doses between about 175 and about 225 mg/m². Some embodiments provide for administration of a taxane at doses between about 60 and about 120 mg/m².

It will be appreciated that polymeric micelle nanoparticle formulations can be similarly provided.

Some embodiments provide methods for administering a taxane to animals or humans via intravascular, oral, intramuscular, cutaneous and subcutaneous routes. Specifically, a taxane composition can be given by any of the following routes, among others: intraabdominal, intraarterial, intraarticular, intracapsular, intracervical, intracranial, intraductal, intradural, intralesional, intralumbar, intramural, intraocular, intraoperative, intraparietal, intraperitoneal, intrapleural, intrapulmonary, intraspinal, intrathoracic, intratracheal, intratympanic, intrauterine, and intraventricular. The emulsions of the present invention can be nebulized using suitable aerosol propellants that are known in the art for pulmonary delivery of lipophilic compounds.

Methods for Making Nanoparticle Formulations

As noted above, the present invention provides nanoparticle formulations. Representative among the nanoparticle formulations are nanoparticle emulsion formulations and nanoparticle polymeric micelle formulations. Methods for making nanoparticle emulsion formulations and nanoparticle polymeric micelle formulations are also provided.

In general, the nanoparticle emulsion formulations of the invention are prepared by forming a solution of the therapeutic agent in a suitable solvent (e.g., organic solvent or solvent combination), adding an aqueous medium to provide a pre-emulsion, (or pre-micelle) and then homogenizing the pre-emulsion to provide the emulsion (or micelle).

The therapeutic agent solution includes, in addition to the therapeutic agent, a solvent or solvent combination suitable to the therapeutic agent to provide the therapeutic agent in solution. In addition to the therapeutic agent and solvent(s), the solution can include one or more surfactant or other materials effective to solubilize the therapeutic agent, or stabilize or otherwise impart favorable properties to the product emulsion.

Suitable solvents useful in the formulations of the invention include solvents that are effective in solubilizing (i.e., dissolving or at least substantially dissolving) the therapeutic agent, and organic solvents that are not miscible with aqueous media used in preparing the emulsion product. In a representative embodiment, the therapeutic agent is a taxol and the solvent is a combination that includes a tocopherol and a polyethylene glycol. In this representative embodiment, the pre-emulsion further includes materials that are effective to stabilize the product emulsion. Representative additional materials include surfactant materials such as a tocopherol polyethylene glycol and a polyoxypropylene-polyoxyethylene glycol nonionic block polymer. In a particular embodiment, the taxol is paclitaxel, the tocopherol is α-tocopherol, the polyethylene glycol is a polyethylene glycol having a molecular weight from about 200 to about 600 g/mole, the tocopherol polyethylene glycol is α-tocopherol polyethylene glycol 1000 succinate, and the polyoxypropylene-polyoxyethylene glycol nonionic block polymer is POLOXAMER 407.

The therapeutic agent solution is the solution that becomes the oil phase of the emulsion. Once formed, the therapeutic agent solution is combined with an aqueous medium to provide the pre-emulsion, which is then homogenized to provide the product emulsion.

The following are representative methods for making paclitaxel nanoparticle emulsions of the invention. In one embodiment, the method for making a paclitaxel/tocopherol-containing emulsion comprises (a) combining paclitaxel and polyethylene glycol to provide a first paclitaxel-containing solution; (b) adding a tocopherol polyethylene glycol (e.g., TPGS) (and optionally a polyoxypropylene-polyoxyethylene glycol nonionic block polymer) to the first paclitaxel-containing solution to provide a second paclitaxel-containing solution; (c) adding a tocopherol to the second paclitaxel-containing solution to provide a third paclitaxel-containing solution, (d) blending the third paclitaxel-containing solution with an aqueous phase to form a pre-emulsion; and (e) homogenizing the pre-emulsion to form an emulsion.

In the practice of the method for making the nanoparticle emulsion formulations of the invention, the pre-emulsion is transformed to the product emulsion by homogenization. Homogenization can be achieved by a variety of devices known in the art including microfluidizers and homogenizers. The desired nanoparticle size can be achieved by a variety of techniques know to the skilled person including microfluidizer and homogenizer operating conditions (e.g., flow rate, pressure, and the number of passes through the device). The desired nanoparticle size can also be achieved by a varying the ratio of organic to aqueous in the pre-emulsion as well as varying the composition of the oil (organic) phase such as components and component amounts (e.g., therapeutic agent load).

The following is a description of a representative homogenization process for the paclitaxel emulsion formulations. It will be appreciated that the homogenization process can be utilized to prepare polymeric micelle nanoparticle formulations. In one embodiment, the pre-emulsion is transferred to the feed vessel of a microfluidizer (e.g., Microfluidizer Model 110Y, Microfluidics Inc, Newton, Mass.). The unit is immersed in a bath to maintain a process temperature of approximately 60° C. during homogenization, and is flushed with argon before use. After priming, the emulsion is passed through the homogenizer in continuous re-cycle for 10 minutes at a pressure gradient of about 18 kpsi across the interaction head. The flow rate is about 300 mL/min, indicating that about 25 passes through the homogenizer result. In another embodiment, the pre-emulsion at 40-45° C. is homogenized in a homogenizer (e.g., Avestin C5 homogenizer, Avestin, Ottawa, Canada) at 26 kpsi for 12 minutes at 44° C. To avoid gelation of the TPGS during the early stage of emulsification, all operations can be performed above 40° C.

A first representative paclitaxel emulsion formulation (10 mg/mL) is prepared as follows: paclitaxel 1.0 gm %, tocopherol 6.0 gm %, TPGS 3.0 gm %, Poloxamer 407 (BASF Corp, Parsippany, N.J.) 1.0 gm %, sorbitol 4.0 gm %, triethanolamine to pH 6.8, and water for injection qs to 100 mL. In this example, 1.0 gm Poloxamer 407 and 1.0 gm paclitaxel were dissolved in 6.0 gm tocopherol with ethanol 10 volumes and gentle heating. The ethanol was then removed under vacuum. Separately, an aqueous buffer was prepared by dissolving 3.0 gm TPGS and 4.0 gm sorbitol in a final volume of 90 mL water for injection. Both oil and water solutions were warmed to 45° C. and mixed with sonication to make a pre-emulsion. A vacuum was used to remove excess air from the pre-emulsion before homogenization. Homogenization was performed in an Avestin C5 homogenizer with the pressure differential across the homogenization valve at 25 kpsi and the temperature of the feed at 42-45° C. A chiller is used to ensure that the product exiting the homogenizer did not exceed a temperature of 50° C. Flow rates of 50 mL/min were obtained during homogenization. After about 20 passes in a recycling mode, the emulsion becomes translucent. Continuing homogenization for 20 min. provides a tocopherol emulsion for intravenous delivery of paclitaxel.

A second representative paclitaxel emulsion formulation (5 mg/mL) is prepared as follows: paclitaxel 0.5 gm %, tocopherol 6.0 gm %, TPGS 3.0 gm %, Poloxamer 407 1.0 gm %, sorbitol 4.0 gm %, triethanolamine to pH 6.8, and water for injection qs to 100 mL. Following homogenization as described above, a translucent emulsion of tocopherol and paclitaxel is obtained.

A third representative paclitaxel emulsion formulation (5 mg/mL) is prepared as follows: paclitaxel 0.5 gm %, tocopherol (synthetic tocopherol USP-FCC, Roche Vitamins Nutley, N.J.) 6.0 gm %, TPGS 3.0 gm %, Poloxamer 407 1.5 gm %, polyethylene glycol 200 (Sigma Chemical Co.) 0.7 gm %, sorbitol 4.0 gm %, triethanolamine to pH 6.8, and water for injection qs to 100 mL. Following homogenization as described above, a translucent emulsion is obtained.

In certain embodiments, the surfactants of the formulation are included in the initially formed, organic therapeutic agent-containing solution, which ultimately becomes the emulsion's oil phase. A representative embodiment is prepared as follows. 1.066 g paclitaxel is dissolved in 12.887 g PEG400 by mixing (low shear at 75° C.); 10.739 g TPGS and 2.157 g Pluronic F127 are added and mixed (low shear) at 50-60° C. until both surfactants are completely melted/dissolved. Then 17.176 g Vitamin E is added and mixed (low shear) at 45-50° C. until the mixture is visibly homogeneous. 21.8 g of the oil phase produced in as described above is added over 1 minute to 79.5 g water while mixing at medium shear (laboratory mixing motor). Mixing is continued for a total of 3 minutes to form a pre-emulsion. The pre-emulsion is homogenized in an Avestin C5 homogenizer in continuous recycle mode for 30 minutes at 22 kpsi peak stroke pressures. From a processing perspective, it can be advantageous to include all of the surfactants in the oil phase.

Methods for Making Targeted Nanoparticle Formulations

In certain aspects, the nanoparticle formulations of the invention are targeted nanoparticle formulations that include a targeting agent associated with the nanoparticle. For nanoparticle formulations, the targeting agent is associated with the nanoparticle (e.g., emulsion oil droplet or polymeric micelle) dispersed in the aqueous phase. In these embodiments, the targeting agent is presented on the exterior of the nanoparticle. Methods for associating the targeting agent to the nanoparticle (e.g., covalent coupling) are known to those of skill in the art. Typically, the targeting agent is covalently coupled to the nanoparticle surface. In these embodiments, the targeting agent is covalently coupled to a component of the nanoparticle that presents or resides on the nanoparticle surface.

For nanoparticle formulations that include a tocopherol polyethylene glycol (e.g., TPGS), a tocopherol polyethylene glycol suitably reactive toward the targeting agent can be employed in the preparation of the emulsion. In certain embodiments, the targeting agent is covalently coupled to the nanoparticle post-nanoparticle formation.

Exemplary tocopherol polyethylene glycols suitable for use in making targeted nanoparticle formulations include carboxy-terminated tocopherol polyethylene glycols. A representative carboxy-terminated tocopherol polyethylene glycol is a carboxy-terminated tocopherol polyethylene glycol succinate.

Representative carboxy-terminated tocopherol polyethylene glycol succinates can be prepared by reacting TPGS (terminal hydroxy group) with a dicarboxylic acid to provide an ester-linked product having a terminal carboxy group (i.e., TPGS-COOH). Briefly, TPGS-COOHs can be prepared by reacting TPGS, a dicarboxylic acid, N,N′-dicyclohexylcarbodiimide (DCC), and dimethylaminopyridine (DMAP) in dimethylsulfoxide (DMSO) at a TPGS/dicarboxylic acid/DCC/DMAP stoichiometric molar ratio of 1:1:1:0.1 (under nitrogen at room temperature for 24 hours). The product TPGS-COOH can be isolated by filtration to remove dicyclohexyl urea (DCU) and then dialyzed against DMSO to remove excess DCC and finally against water to remove DMSO. A variety of dicarboxylic acids can be employed. In one embodiment, the dicarboxylic acid is glutaric acid.

Once the nanoparticle is formed using TPGS-COOH as a component of the nanoparticle, the targeting agent is associated with the nanoparticle. Suitable targeting agents include a functional group that is reactive toward the nanoparticle's surface carboxyl group imparted by the TPGS-COOH. The targeting agent's reactive group (e.g., amino group, —NH₂) may be native to the targeting agent or incorporated into the targeting agent by methods known to those of skill in the art.

In a representative procedure, the surface carboxyl groups of the nanoparticles are activated by N-hydroxysuccinimide (NHS) and 1-ethyl-3-(3-dimethylamino)propyl carbodiimide (EDC) at a TPGS-COOH/EDC/NHS stoichiometric molar ratio of 1:20:6.7 (under nitrogen at room temperature for 2 hours). The activated nanoparticles are then treated with targeting agent (i.e. NH₂-targeting agent) at a TPGS-COOH/NH₂-targeting agent stoichiometric molar ratio of 1:10 followed by pH adjustment to about 8 (e.g., 4 hours at 37° C.) to provide nanoparticles labeled with the targeting agent.

It will be appreciated that the desired number of targeting agents per nanoparticle can be varied depending on the nature of the nanoparticle and targeting agent, and can be obtained by varying reaction conditions (e.g., reactant stoichiometry of reactants) as known by those of skill in the art.

Representative targeting agents useful for making targeted nanoparticle formulations are described above. In certain embodiments, the targeting agent is an antibody or antibody fragment having an affinity to cancer cell surface markers.

While the preferred embodiment of the invention has been illustrated and described, it will be appreciated that various changes can be made therein without departing from the spirit and scope of the invention.

1. A nanoparticle, comprising:
(a) a core comprising a therapeutic agent and a tocopherol; and
(b) a shell comprising a tocopherol polyethylene glycol derivative,
wherein the shell surrounds the core, and
wherein the nanoparticle has a size sufficient to be taken into and/or across a human cell by active transport through a cell surface organelle.

2. The nanoparticle of claim 1, wherein the therapeutic agent is a taxane.

3. The nanoparticle of claim 1, wherein the therapeutic agent is paclitaxel.

4. The nanoparticle of claim 1, wherein the tocopherol is selected from the group consisting of α-tocotrienol, β-tocotrienol, γ-tocotrienol, δ-tocotrienol, α-tocopherol, α-tocopherol acetate, α-tocopherol phosphate, α-tocopherol succinate, α-tocopherol nicotinate, and α-tocopherol linoleate.

5. The nanoparticle of claim 1, wherein the tocopherol is α-tocopherol.

6. (canceled)

7. The nanoparticle of claim 1, wherein the tocopherol polyethylene glycol derivative is D-α-tocopherol polyethylene glycol 1000 succinate.

8-9. (canceled)

10. The nanoparticle of claim 1 further comprising polyethylene glycol.

11. The nanoparticle of claim 1 further comprising a polyoxypropylene-polyoxyethylene glycol nonionic block copolymer.

12. (canceled)

13. The nanoparticle of claim 1, wherein the nanoparticle is an emulsion droplet.

14. The nanoparticle of claim 1, wherein the nanoparticle is a polymeric micelle.

15. The nanoparticle of claim 1 further comprising a targeting agent.

16. The nanoparticle of claim 15, wherein the targeting agent is selected from the group consisting of small molecules, peptides, proteins, aptamers, and nucleic acids.

17. The nanoparticle of claim 15, wherein the targeting agent is an antibody or functional fragment thereof.

18. (canceled)

19. The nanoparticle of claim 1 further comprising an imaging agent.

20-21. (canceled)

22. An formulation, comprising:
(a) a first phase; and
(b) a second phase comprising a plurality of nanoparticles of claim 1.

23-26. (canceled)

27. A method for treating a disease, condition, or disorder treatable by administering a specific therapeutic agent, comprising administering to a subject in need thereof a therapeutically effective amount of a nanoparticle of claim 1.

28. The method of claim 27, wherein the therapeutic agent is taken into and/or across a targeted cell by active transport through a cell surface organelle.

29. The method of claim 27, wherein the therapeutic agent is taken into and/or across a targeted cell by active transport through caveolin-mediated endocytosis.

30. The method of claim 27, wherein the therapeutic agent is taken into and/or across a targeted cell by active transport through clathrin-mediated endocytosis.

31. The method of claim 27, wherein the therapeutic agent is taken into and/or across a targeted cell by active transport through caveolin- and clathrin-independent endocytosis.

32-41. (canceled)
